(19) United States
(12) Patent Application Publication
(10) Pub. No.: US 2024/0065751 A1
(43) Pub. Date: Feb. 29, 2024

(54) ABLATION PROBE AND LUMEN FOR IMPROVED ACCESS AND FLOW

(71) Applicant: Arthrex, Inc., Naples, FL (US)

(72) Inventors: Jeffrey Haczynski, Naples, FL (US); Richard J. Taft, Naples, FL (US); Stephen Donnigan, Castle Rock, CO (US); Jeremiah D. Caldwell, Napel, FL (US)

(73) Assignee: Arthrex, Inc, Naples, FL (US)

(21) Appl. No.: 18/232,487

(22) Filed: Aug. 10, 2023

Related U.S. Application Data
(60) Provisional application No. 63/400,900, filed on Aug. 25, 2022.

(51) Int. Cl. A61B 18/14

(52) U.S. Cl. CPC A61B 18/148; A61B 2018/00577; A61B 2218/002; A61B 2218/007

(57) ABSTRACT

An apparatus and method provide for the delivery of an ablation treatment by supplying a control signal to supply electrode of an ablation device disposed at the distal end portion of an elongated shaft. A lumen extends along a longitudinal axis of the shaft. The supply electrode forms an electrode face laterally directed from the longitudinal axis and through which an aperture extends in connection with the lumen. The electrode face forms an ovular shape having a first major axis that extends parallel to the longitudinal axis. An insulator separates the supply electrode from a return electrode, and a transition passage of the lumen extends though the insulator along an arc-shaped path.

### CROSS-REFERENCE TO RELATED APPLICATIONS

This application claims priority under 35 U.S.C. § 119(e) and the benefit of U.S. Provisional Application No. 63/400,900 entitled ABLATION PROBE AND LUMEN FOR IMPROVED ACCESS AND FLOW, filed on Aug. 25, 2022, by Jeffrey Haczynski et al., the entire disclosure of which is incorporated herein by reference.

### BACKGROUND OF THE INVENTION

The present disclosure generally relates to an ablation apparatus or probe comprising one or more electrodes and a lumen for fluid transmission and more particularly to an ablation probe with improved fluid transmission while maintaining a narrow access envelope. In general, ablation apparatuses may be implemented in minimally invasive surgical operations. Such operations may limit trauma and tissue damage associated with various surgical procedures, thereby improving patient outcomes and limiting patient recovery time.

### SUMMARY

In general, the disclosure provides for ablation devices designed to deliver high frequency signals to one or more electrodes to remove or manipulate tissue associated with a surgical procedure. Such devices may include various designs configured to access cavities or internal anatomical features of patients by being implemented as distally positioned accessories in connection with elongated probes. However, the design and operation of ablation probes may commonly result in clogging of fluid transmission passages or lumens as well as proportions that create challenges in accessing anatomical features or cavities. Challenges may further be exacerbated in cases where cannulas or access ports having narrow interior passageways or access envelopes are implemented in procedures. The disclosure generally provides for an ablation apparatus and corresponding features to improve electrode operation and access by limiting proportions of an exterior profile shape, such that the ablation probe may be utilized to enter and access a long, narrow access envelope. In combination with the improved electrode operation and streamlined profile shape, the disclosed ablation probe may further provide for an improved fluid transmission passage or lumen extending through an elongated shaft or body.

In various embodiments, the disclosure provides for an ablation apparatus or probe that may provide various combinations of electrode features to improve ablation operation while incorporating a narrow distal profile to maintain access within surgical sites having limited access envelopes. In some cases, the ablation probe may additionally provide for an internal lumen comprising an interior transition section for improved fluid flow. The interior transition section or transition passage may be applied in combination with the tapered distal end to maintain joint access while also improving fluid flow through one or more apertures or aspiration ports. Accordingly, the disclosure may provide for an ablation probe that improves access by maintaining a tapered profile shape while also improving the delivery of targeted electrical energy and maintaining effective fluid flow through the lumen to limit clogging. The specific features and combinations associated with the beneficial operation of the ablation device are described in the following detailed description.

In some implementations, the disclosure may provide for an ablation apparatus comprising an elongated shaft including a lumen extending along a longitudinal axis from a proximal end portion to a distal end portion. An active electrode may form an electrode face directed laterally from the longitudinal axis at the distal end portion of the elongated shaft and comprising at least one aperture or aspiration port in connection with the lumen. A return electrode may extend along the distal end portion of the elongated shaft and, in some cases, may be approximately equidistant from the active electrode along a distal extent of the ablation apparatus. An insulator may be interposed between the active electrode and the return electrode. In various implementations, the insulator may form an interior passage providing a transition section from the at least one aperture in the active electrode to the lumen extending through the elongated shaft. In various implementations, the interior passage extending through the insulator may provide for a gradual reduction in cross section extending along an arcuate path from the active electrode to the lumen in the elongated shaft.

These and other features, objects and advantages of the present disclosure will become apparent upon reading the following description thereof together with reference to the accompanying drawings.

### BRIEF DESCRIPTION OF THE DRAWINGS

FIG. 1 is a projected view of an ablation device demonstrating an electrode assembly;

FIG. 2A is a front view of an ablation device demonstrating an electrode assembly;

FIG. 2B is a side view of an ablation device demonstrating a profile shape configured to enter an access envelope;

FIG. 3 is a front detailed view of an active electrode of an ablation device;

FIG. 4 is a projected view of the active electrode demonstrated in FIG. 3;

FIG. 5 is a side cross-sectional view demonstrating a lumen of an ablation device;

FIG. 6 is a side cross-sectional view of the lumen introduced in FIG. 5 demonstrating a simulated fluid flow passing through the lumen;

FIG. 7A is a side profile view of an ablation device demonstrating an access envelope;

FIG. 7B is a projected, three-dimensional representation of the access envelope introduced in FIG. 7A;

FIG. 8A is a front view of an ablation device demonstrating an electrode assembly;

FIG. 8B is a side view of an ablation device demonstrating a profile shape configured to enter an access envelope; and

FIG. 9 is an illustrative diagram of an electro-surgical ablation system for use with an ablation device in accordance with the disclosure.

### DETAILED DESCRIPTION OF THE PREFERRED EMBODIMENT

Ablation devices and corresponding systems may provide beneficial utility for minimally invasive medical procedures. Such procedures may limit patient recovery times and improve outcomes by applying specialized surgical techniques and tools to remotely access various treatment areas. As discussed in the following description, the disclosure provides for an ablation device or apparatus configured to effectively deliver ablation treatment to highly constrained anatomical areas. In some examples, the ablation device may provide for an interior lumen configured to provide improved fluid transmission to limit clogging while simultaneously limiting the proportions of an access envelope necessary for the ablation device to reach a cavity or treated area of a patient. In an exemplary embodiment, a tapered or torpedo-shaped distal profile of the ablation device may be implemented with a complimentary electrode configuration and fluid transmission lumen to provide a combination of improved delivery of surgical energy and access while limiting issues with clogging and fluid transmission. Though discussed in relation to specific exemplary devices, each of the corresponding features may be implemented alone or in various combinations to enhance and improve the operation of an ablation device. Accordingly, the disclosure may provide for an improved ablation device or probe suited to a variety of applications.

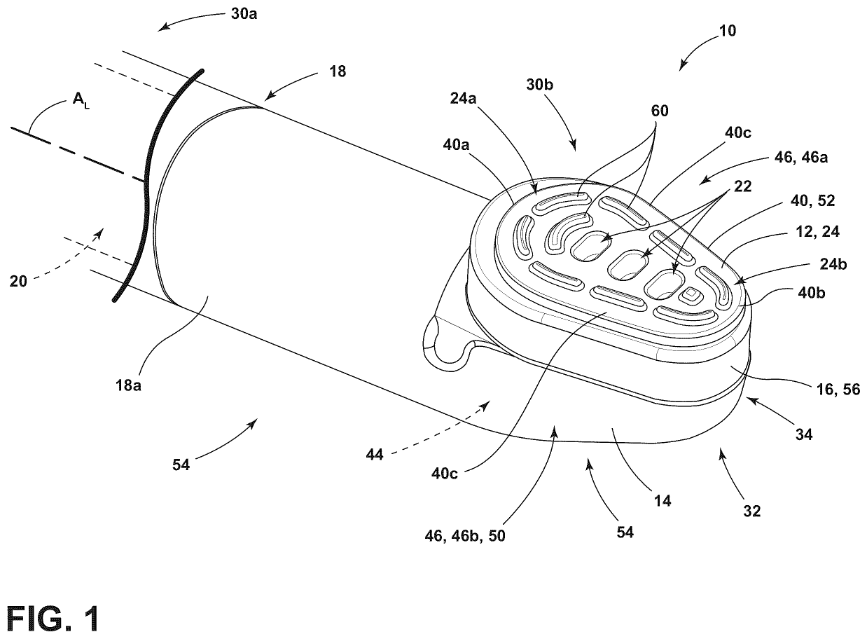

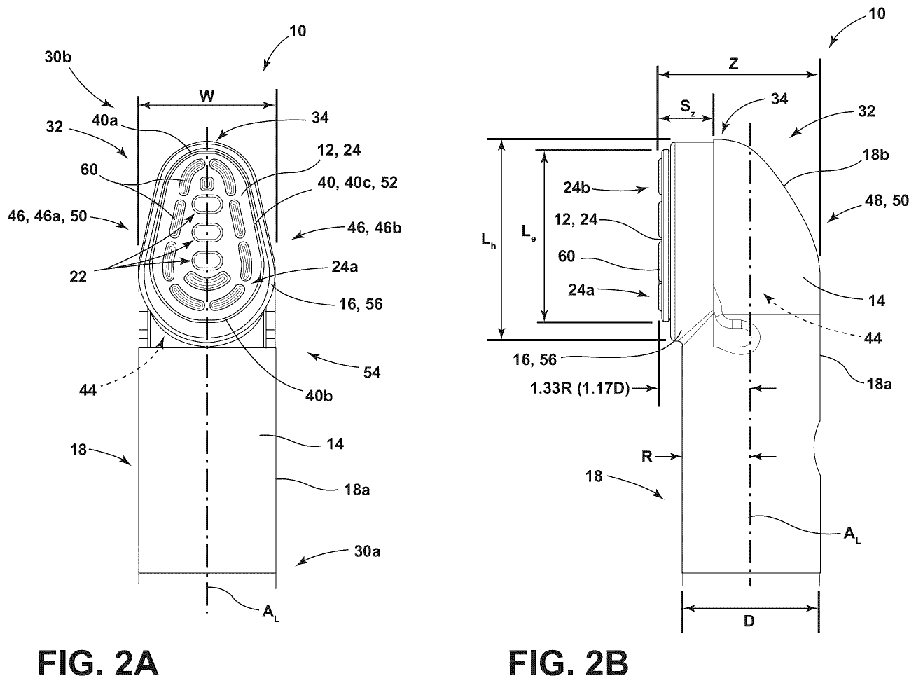

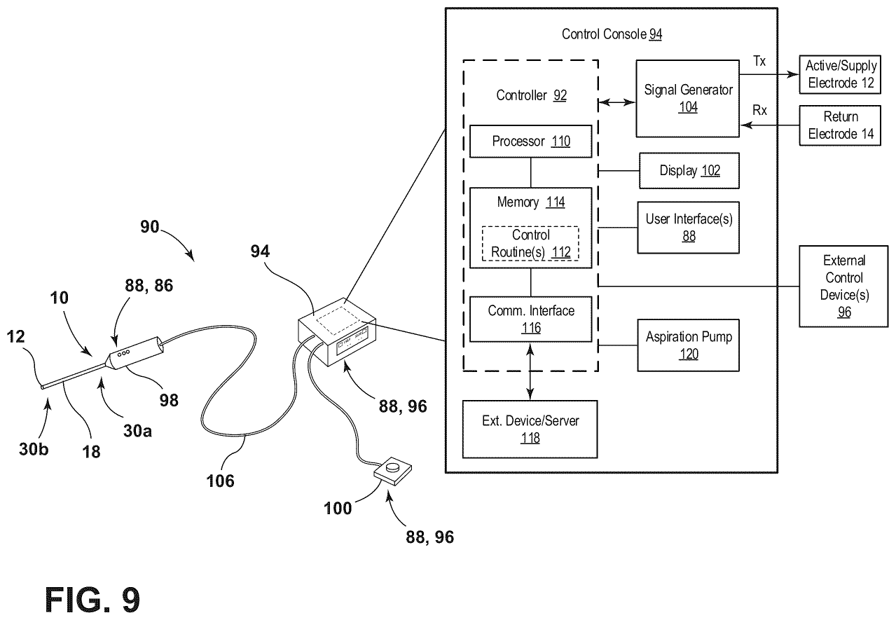

Referring now to FIGS. 1, 2A and 2B, an exemplary ablation device 10 is shown demonstrating an active or supply electrode 12 insularly separated from a passive or return electrode 14 by an insulator 16. As discussed herein, the ablation device 10 may be referred to as an ablation probe, ablation catheter, or more generally as an electro-surgical device. In various implementations, the ablation device 10 may comprise an elongated body or shaft 18 that may provide for an interior passage or a lumen 20 configured to communicate fluid through at least one aspiration port or aperture 22 disposed in an electrode face 24 of the supply electrode 12. As discussed later in detail and demonstrated generally in FIG. 9, the shaft 18 may be in connection with a handle comprising a user interface and may further be in communication with a control console, generally referred to as a controller. Accordingly, the ablation device 10 may provide for the delivery of therapeutic signals (e.g., high frequency or radio frequency signals) configured to treat, remove, or manipulate tissue of patients during surgical procedures. In various implementations, the elongated body of the shaft 18 may provide for the supply electrode 12 to access various cavities or treatment areas within an anatomy of a patient.

The ablation device 10 may extend from a handle or user interface portion, referred to herein as a proximal end portion 30a, to a distal end portion 30b. The distal end portion 30b may correspond to an acting end where the supply electrode 12 is disposed and configured to supply therapeutic energy to a target region of a patient. To ensure that the ablation device 10 is capable of reaching a target area, particularly within the tight confines of a rigid joint space, the distal end portion 30b may comprise a torpedo-like or tapered end portion 32. The tapered end portion 32 may gradually narrow circumferentially about the electrode face 24, which may be substantially flat or planar. The tapering of the distal end portion 30b may narrow distally along a longitudinal axis A_{L} circumferentially about the electrode face 24 in a parabolic dome along at least a portion of a length of the supply electrode 12. The distal end portion 30b of the ablation device 10 may terminate at a distal extent 34. In general, the tapered end portion 32 of the ablation device 10 may provide for the distal end portion 30b to access various cavities or openings within the anatomy of a patient without encountering challenges or encumbrances due to protrusions extending outward or bending away from the longitudinal axis A_{L}.

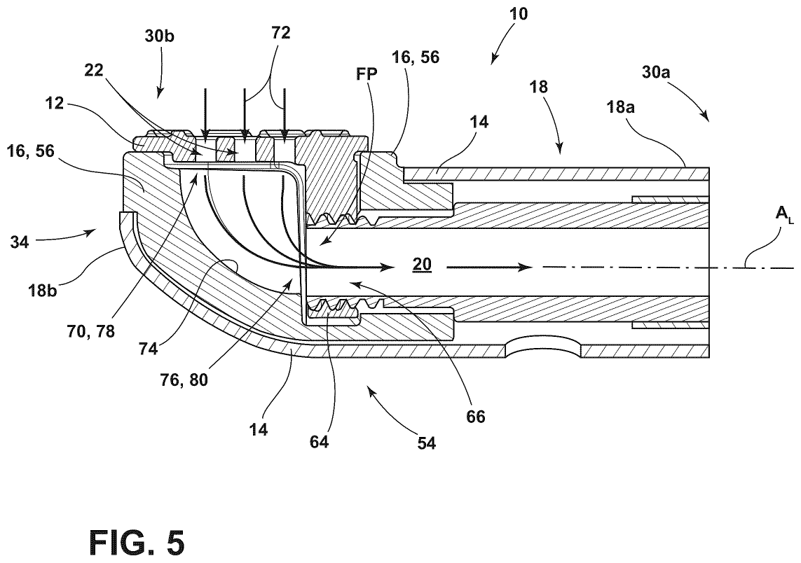

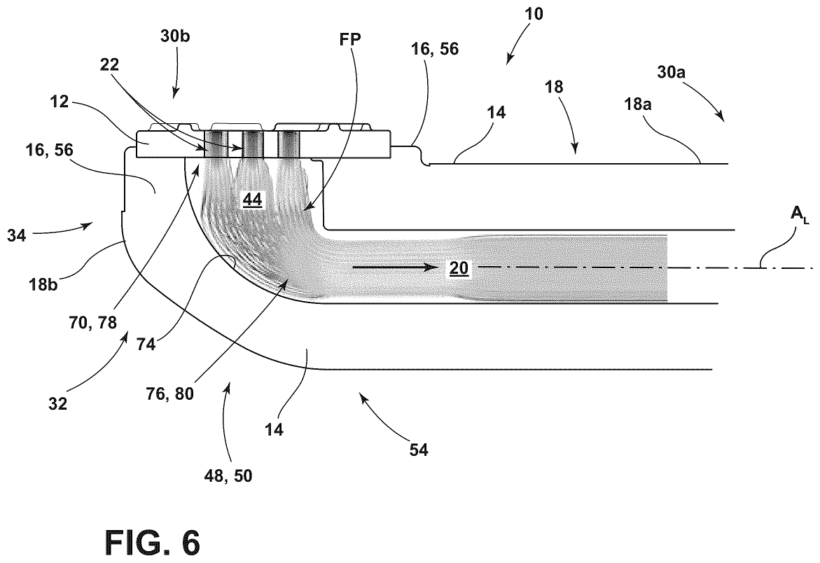

As discussed further in various examples that follow, the tapered end portion 32 of the ablation device 10 may be implemented in combination with the ovular profile shape 40 of the supply electrode 12. In such implementations, the profile shape 40 may narrow in parallel with the tapered end portion 32 of the insulator 16 to improve the distal access of the device 10. In general, the supply electrode 12 may be configured to effectively deliver radio frequency (RF) energy between the supply electrode 12 and the return electrode 14, even over the distal extent 34 of the device 10. As further discussed in reference to FIGS. 5A, 5B, and 6, the insulator 16 or distal body of the ablation device 10 may form an interior passage in connection with or forming a distal end portion of the lumen 20, which may be referred to as an interior transition section or transition passage 44. The transition passage 44 may provide for a smooth swept path between the one or more aspiration ports 22 disposed in the supply electrode 12 and the lumen 20 extending along the longitudinal axis A_{L} of the shaft 18. The transition passage 44 may provide for a smooth arcuate transition from the path of the aspiration ports 22 perpendicular to the longitudinal axis A_{L} to the lumen 20 or central passage through the shaft 18, which is aligned parallel to the longitudinal axis A_{L}. Accordingly, the profile shape 40 of the supply electrode 12 may be implemented in combination with the transition passage 44 of the lumen 20, such that the tapered end portion 32 of the ablation device 10 may be effectively implemented without impeding fluid transmission through the lumen 20.

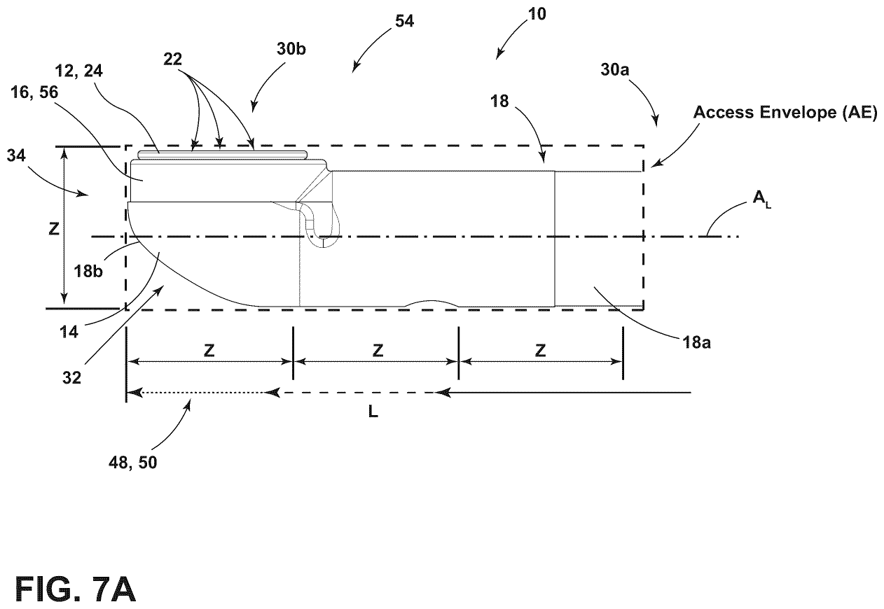

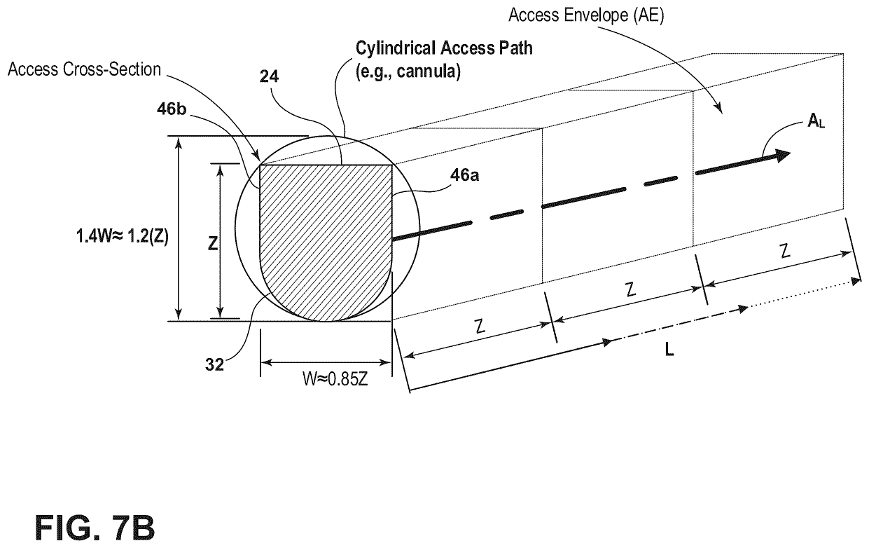

The tapered end portion 32 and corresponding features of the ablation device 10 may provide for the electrode face 24 to be substantially planar, having a projecting surface directed laterally from the longitudinal axis A_{L} while limiting protrusions or spatial divergences away from the longitudinal axis A_{L}. In this way, an elongated body of the ablation device 10 may enter a long and narrow access envelope (AE), such that the ablation device 10 may be implemented in a variety of endoscopic or arthroscopic procedures. An exemplary representation of the access envelope AE is shown in FIGS. 7A and 7B and may visually represent the long, narrow confines associated with joint access through a cannula or long, narrow patient access port. In various embodiments, the ablation device 10 may incorporate the features of the profile shape 40 of the supply electrode 12, the transition passage 44 of the lumen 20, and/or the tapered end portion 32 of the insulator 16 to provide effective delivery of the RF energy to the supply electrode 12 while maintaining a compact package that can easily enter a long narrow passage exemplified by the access envelope AE. Each of the corresponding features of the ablation device 10 are further discussed in the following examples. Though discussed in reference to specific examples and exemplary combinations, it shall be understood that the beneficial features of the ablation device 10 described herein may be applied in various combinations or alone to provide improved access and operation for various surgical applications.

Referring still to FIGS. 1, 2A, and 2B, the tapered end portion 32 may extend along or parallel to opposing sides 46 of a head portion 50 of the ablation device 10. In this configuration, a first side 46a and a second side 46b of the head portion 50 may progressively taper along a perimeter edge 52 formed by the profile shape 40 of the supply electrode 12. As illustrated in FIG. 2A, the head portion 50 as well as the profile shape 40 of the supply electrode 12, may extend longitudinally along an elongated oval shape or ovular face forming the profile shape 40 and having a major axis that extends parallel to the longitudinal axis A_{L}. The electrode face 24 may comprise a proximal electrode portion 24a and a distal electrode portion 24b aligned with the proximal end portion 30a and the distal end portion 30b of the ablation device 10, respectively. The profile shape 40 extending along the proximal electrode portion 24a may form a first arc 40a comprising a first radius. The profile shape 40 of the electrode face 24 extending along the distal electrode portion 24b may form a second arc 40b having a second radius that is smaller than the first radius of the first arc 40a. Between the proximal and distal electrode portions 24a, 24b, the profile shape 40 may include straight sides 40c extending along converging paths from the first arc 40a to the second arc 40b. As shown, the tapered end portion 32 of the distal body or insulator 16 of the ablation device 10 may extend approximately parallel and equidistant to the straight sides 40c and the second arc 40b of the profile shape 40 of the supply electrode 12. In this configuration, the profile shape 40 of the supply electrode 12 may define the tapered profile shape of the body for insulator 16 forming the opposing sides 46 of the tapered end portion 32.

As best depicted in FIG. 2B, opposite the electrode face 24, a rear surface 48 of the head portion 50 may taper inward from an outer wall 18a of the shaft 18, which extends along the longitudinal axis A_{L}, from a longitudinal extent of the ablation device 10 aligned with the first arc 40a to the distal extent 34. The tapered end portion 32 or profile shape of the distal body of the ablation device 10 may gradually curve from the outer wall 18a along a parabolic or complex arcuate path toward the longitudinal axis A_{L}. In this configuration, the tapered end portion 32 or tapered profile shape depicted in FIG. 2B may form a distal wall 18b that converges circumferentially on the longitudinal axis A_{L} in coordination with the profile shape 40 of the supply electrode 12 depicted in FIG. 2A to the distal extent 34 of the ablation device 10. As discussed later in reference to FIGS. 5 and 6, the tapered end portion 32 may provide space for or accommodate the arcuate shape and proportions of the transition passage 44 of the lumen 20 passing through the distal body of the ablation device 10 as formed by the insulator 16 in the exemplary implementation. Accordingly, an acting end 54 of the ablation device 10, comprising the supply electrode 12, the insulator 16, and the return electrode 14, may provide for a combination of beneficial operation of high frequency radio signals within an access envelope AE while also providing improved fluid flow through the interior lumen 20 and transition section 44.

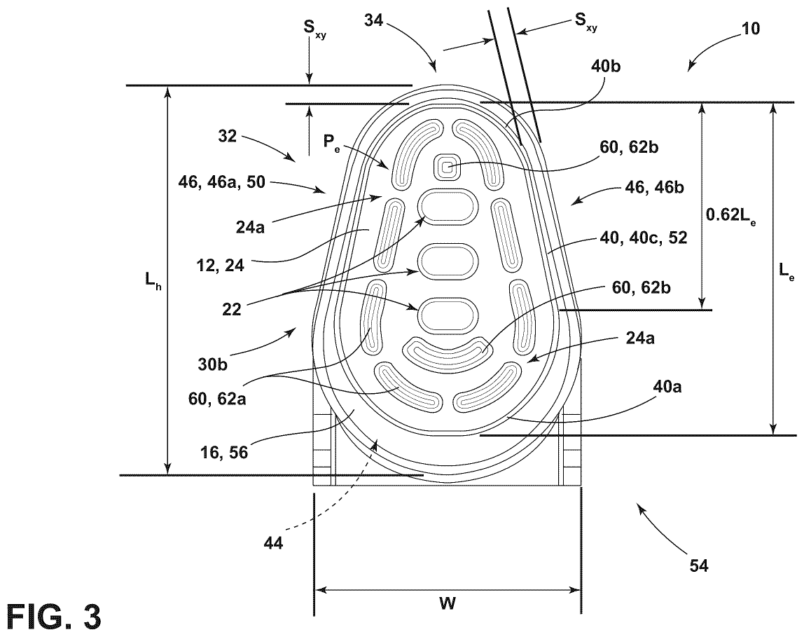

Referring now to FIGS. 2B and 3, the profile shape 40 of the supply electrode 12 is discussed in reference to a spacing or separation between the supply electrode 12 and the return electrode 14 formed by an exposed portion 56 the insulator 16. As shown, a length of the supply electrode 12 is denoted as the electrode length Lₑ. The second arc 40b and straight sides 40c forming the profile or ovular shape 40 of the supply electrode 12 may extend along approximately 0.6(Lₑ) (e.g., 0.62(Lₑ)) or a distal 60% of the electrode length Lₑ. Accordingly, the proximal electrode portion 22a may extend along the electrode length Lₑ over a proximal 40% (e.g., 0.38(Lₑ)) of the total electrode length Lₑ. FIG. 3 further demonstrates a head length Lₕ denoting a longitudinal length of the head portion 50 along the longitudinal axis A_{L} over which the electrode length Lₑ extends. As shown, the electrode length Lₑ is biased toward the distal extent 34 of the ablation device 10, and the perimeter edge 52 of the supply electrode 12 is spaced apart approximately equidistant from the return electrode 14 by the insulator 16 along the distal electrode portion 24b of the supply electrode 12. As demonstrated in FIGS. 2B and 3, the spacing along the electrode face 24 is denoted as S_{xy} and the spacing perpendicular to the electrode face 24 is denoted as S_{z}. As shown, the lengths of S_{xy} and S_{z} remain consistent over the distal electrode portion 24b. As shown, the spacing (S_{xy}, S_{z}) between the perimeter edge 52 of the supply electrode 12 and the return electrode 14 remains approximately constant or equidistant along the distal 60% of the electrode length Lₑ. Though the distal 60% electrode length Lₑ of the supply electrode 12 is shown having the even or approximately constant spacing (S_{xy}, S_{z}) from the return electrode 14, the approximately constant spacing (S_{xy}, S_{z}) may extend along approximately 90% of the electrode length Lₑ to as little as 10% of the electrode length Lₑ or more specifically along at least a distal 10%, 25%, 40%, 55%, 70%, or 85% of the electrode length Lₑ.

As discussed herein, the term “approximate” may correspond to and include minor variations in dimensions that may be associated with equivalent structures as well as variations in various manufacturing processes. For example, as discussed herein, the spacing being approximately equidistant or constant may correspond to and include variations in spacing (S_{xy}, S_{z}) ranging from approximately 5% to 10% between the supply electrode 12 and the return electrode 14. Similarly, approximately constant spacing may correspond to spacing that is nearly constant about the perimeter edge 52 of the supply electrode 12 on average, which may incorporate various dimensional variations about the perimeter edge 52 while still maintaining an equivalent average dimensional spacing therebetween. In general, the term “approximate” may be used in this application to describe various relationships and/or dimensions and may be interpreted to include corresponding variations that may provide for similar or equivalent structures. Accordingly, dimensional or relational variations of 5% to 10% may be associated with the use of approximate terminology herein. The extent of the variation or range associated with the terms approximate or substantially may be understood by those skilled in the art based on the nature of the shapes, dimensions, relationships and the corresponding structures, features, and applications to which they correspond, such that the metes and bounds are limited to structures that maintain the operational functionality associated with the particular technological solution associated.

Though discussed in reference to the specific illustrated embodiment shown in FIG. 3, it shall be understood that the constant or approximately equidistant spacing between the supply electrode 12 and the return electrode 14 may similarly extend over approximately 25%, 40%, or 55% of the distal electrode length Lₑ of the supply electrode 12. In such cases, a spacing between the distal electrode portion 24b of the supply electrode 12 may be increased about the perimeter edge 52 of the supply electrode 12. Accordingly, the taper or circumferential convergence of the tapered end portion and the corresponding tapered profile shape of the insulator 16 may be less severe (e.g., less sloped toward the longitudinal axis A_{L}) than demonstrated in the example shown. For example, in some instances, the head portion 50 may extend further from the perimeter edge 52 along the straight sides 40c of the profile shape 40. In each of the examples described, the position of the perimeter edge 52 of the electrode face 24 and corresponding approximately equidistant or constant spacing from the return electrode 14 about the distal electrode portion 24b may provide for consistent transmission of high frequency electrical energy from the supply electrode 12 to generate a consistent ablative effect across the insulator 16 to the return electrode 14. This spacing, particularly when applied in combination with one or more surface features 60 incorporated on the electrode face 24, may provide for consistent and evenly distributed communication of the ablation energy along the distal electrode portion 24b of the supply electrode 12.

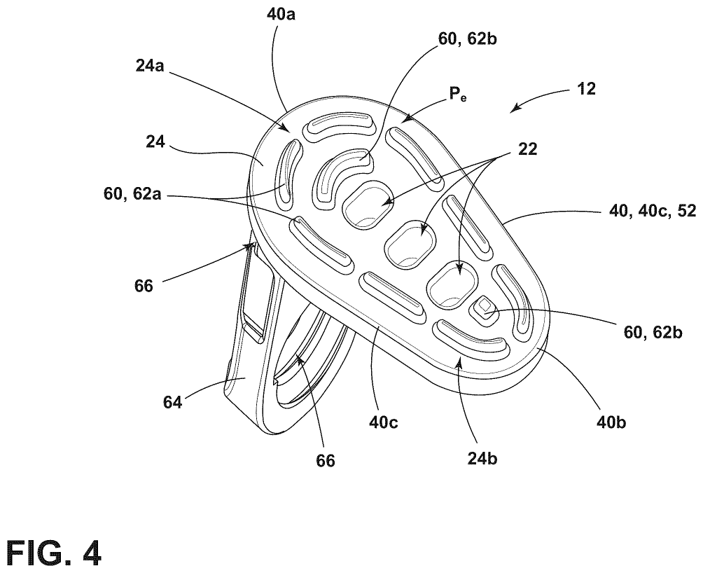

Referring now to FIGS. 3 and 4, surface features of the electrode face 24 may include one or more raised ridges or protrusions 62 extending outward from the electrode face 24 or laterally from a corresponding electrode plane P e extending parallel to and laterally offset from the longitudinal axis A_{L} over the electrode face 24. The protrusions 62 may correspond to a plurality of rounded ridges that may extend substantially parallel to the perimeter edge 52 of the supply electrode 12. For example, the protrusions 62 may comprise a first group of raised ridges 62a that extend parallel to or approximately parallel to the perimeter edge 52 of the supply electrode 12. Additionally, the first group of raised ridges 62a may correspond to a plurality of segments approximately equal in length and evenly distributed about the perimeter edge 52 of the supply electrode 12. As shown, the first group of raised ridges 62a of the protrusions 62 may correspond to a segmented perimeter ridge that extends about a second group of raised ridges 62b as well as the one or more aspiration ports 22 for apertures formed in the supply electrode 12.

The at least one aspiration port 22 may correspond to a plurality of aspiration ports 22 (e.g., three aspiration ports), which may be evenly spaced over the electrode face 24 within a perimeter formed by the first group of raised ridges 62a and the second group of raised ridges 62b or, more generally, the protrusions 62. As further discussed in reference to FIGS. 5 and 6, the plurality of apertures or aspiration ports 22 are formed centrally through the supply electrode 12 and aligned within an internal cross section of a passage formed by the transition passage 44 in connection with the lumen 20. In this configuration, the aspiration ports 22 may draw a substantially even distribution of fluid from an operating region or cavity through the aspiration ports 22 and supply a laminar flow through the transition passage 44 in communication with the lumen 20. The second group of raised ridges 62b may be positioned between the first group of raised ridges 62a and the aspiration ports 22 along the proximal electrode portion 24a and the distal electrode portion 24b. More specifically, the second group of raised ridges 62b may be centrally positioned between the perimeter formed by the first group of raised ridges 62a along or parallel to the perimeter edge 52 of the supply electrode 12. The second group of raised ridges 62b may further be disposed approximately centrally on the electrode face 24 between the first group of raised ridges 62a and the plurality of aspiration ports 22.

As demonstrated in FIG. 4, a mounting collar 64 may be connected to a mounting surface 66 of the supply electrode 12 and configured to connect to the insulator 16 and a supply terminal in connection with the control console. The mounting collar 64 may extend from the mounting surface 66 on the proximal electrode portion 24a. The mounting collar 64 may further form an opening through which the lumen 20 of the shaft 18 may interconnect with the supply electrode 12 and interconnect the lumen 20 to the transition passage 44 formed through the insulator 16 or the distal body forming the head portion 50 of the ablation device 10. In addition to being evenly distributed over the electrode face 24, the aspiration ports 22 may each form elongated ovular shapes that may include a major axis of the ovular profile shape 40 of the supply electrode 12. Similarly, the major axis of the ovular shapes forming the aspiration ports 22 may extend perpendicular to a major axis or elongated dimension of the cross section forming an inlet cross section 78 (see FIG. 5) of the interior transition section or transmission passage 44. In this configuration, the fluid passing through the aspiration ports 22 may be centrally delivered within an inlet end 70 of the interior transition section or interior passage 44 to improve flow distribution and provide laminar flow through the transition section 44 and into the lumen 20. The interface between the collar opening 68, the lumen 20, and the transition passage 44 is further demonstrated and discussed in reference to FIG. 5.

Referring now to FIGS. 5 and 6, a fluid flow path FP (denoted by the arrows in FIG. 5) is discussed in reference to the lumen 20, the transition section 44, and the aspiration ports 22 of the ablation device 10. As previously discussed, the tapered end portion 32 formed by the rear surface 48 of the head portion 50 is shown in the cross section of FIG. 5 relative to the transition passage 44 of the head portion 50 formed by the insulator 16. Additionally, the aspiration ports 22 are demonstrated as being evenly distributed across the opening formed along an inlet end 70 formed by the transition passage 44. In some implementations, the transition section 44 may comprise an arc-shaped path swept from an inlet direction (denoted as arrows 72) to the longitudinal axis A_{L}, oriented approximately perpendicular to or 90 degrees to the inlet direction 72. A swept arc 74, along which the interior transition passage or section 44 extends through the distal body or insulator 16 of the ablation device 10, may provide for an interior cross section of the supply electrode 12 that gradually decreases from the inlet end 70 to an outlet end 76 in connection with lumen 20. More specifically, the interior cross section (e.g., the surface extending inward and outward from the page as depicted in FIG. 5) may be gradually decreased due to the transition of the interior passage formed by the transition section 44 along the swept arc 74. As shown, the swept arc 74 may provide for an inlet cross section 78 of the transition section or passage 44 at the inlet end 70 to be at least 20% larger than an outlet cross section 80 of the transition section 44 at the outlet end 76. Additionally, the outlet cross section 80 may be approximately equivalent to or equal in cross-sectional area to the interior passage formed by the lumen 20 along the shaft 18. In various embodiments, the surface area associated with the inlet cross section 78 formed by the transition passage or transition section 44 may vary based on the design of the supply electrode and the corresponding distribution of the aspiration ports 22. For example, in some cases, the inlet cross section 78 may be approximately 30%, 40%, 50%, 60%, 70%, or even 80% larger than the outlet cross section 80 of the transition section 44. As depicted in the example in FIG. 5, the inlet cross section 78 is 82% larger than the outlet cross section 80. By providing the increased cross-sectional area of the inlet cross section 78 gradually reduced over the perpendicular transition along the swept arc 74 to the outlet cross section 80, the ablation device 10 may provide for a gradual increase in flow rate and intensity from the inlet cross section 78 to the lumen 20. The gradual transition may limit turbulent flow and maintain laminar flow, thereby limiting clogging of the lumen 20.

Referring now to FIG. 6, a flow simulation is shown demonstrating a flow intensity of fluid passing through the aspiration ports 22, into the transition section or transition passage 44, and into the lumen 20 extending along the length of the shaft 18. In particular, the flow simulation demonstrates a peak fluid velocity within the aspiration ports 22. Additionally, the lines demonstrating the flow path FP of the fluid are generally aligned with the swept arc 74 extending through the transition section or transition passageway 44 from the supply electrode 12 to the lumen 20. The consistent alignment of the lines associated with the flow simulation as opposed to swirling or misaligned trajectories demonstrate a laminar flow extending from the aspiration ports 22 through the transition section 44 and into the lumen 20. The laminar flow through the ablation device 10 may be provided by the geometry of the transition passage 44, which limits turbulence and associated clogging. In general, the consistent velocity and laminar flow along the perpendicular transition through the swept arc 74, as well as the gradual decrease of the associated cross-sectional area between the inlet cross section 78 and the outlet cross section 80, may ensure that a consistent fluid velocity maintains directional transmission of particles within the lumen 20 to prevent clogging.

Though no numeric scale is shown in relation to the flow simulation in FIG. 6, the gradual variations in velocity and consistent flow through the transition passage 44 are apparent based on the consistent shading of the flow lines along the fluid flow FP. The peak velocity of the fluid flow may occur in the aspiration ports 22 and is illustrated by the darkened shading therethrough. The velocity of the fluid demonstrated in the simulation may decrease along an elongated path of the swept arc 74 extending through the transition passage 44. However, the decreased velocity gradually changes and darkens along the swept arc 74, which illustrates a lack of stalled fluid. In contrast, stalled fluid or variations in velocity would be illustrated by abrupt dark spots or regions, which are not apparent. The remainder of the simulated flow data, demonstrated by the shaded lines extending through the lumen 20, is consistent as represented by the limited variations in the gray shading with no evident dark regions (e.g., eddies or low velocity regions). Accordingly, the simulated results in FIG. 6 demonstrate a laminar flow profile that may limit clogging in the transition passage 44 and the lumen 20.

Referring now to FIGS. 7A and 7B, an axis envelope AE is shown and discussed demonstrating an interior passage through which the ablation device 10 may be passed or maneuvered to simulate small openings within the anatomy of a patient. As previously discussed, the ablation device 10 may be implemented as an elongated probe that may be passed into various rigid cavities (e.g., joint cavities) and introduced into the body of a patient through a port or cannula. As shown, the access envelope AE simulates and provides dimensional relationships for volumetric passages through which the ablation device 10 may easily access tissue or areas of interest to provide treatment to a patient. As shown, the dimensions associated with the access envelope are demonstrated relatively in reference to the proportions of the ablation device 10 focusing on an overall height or depth Z of the shaft 18 in combination with the supply electrode 12 as well as a width W defined as the width of the shaft 18 perpendicular to the depth Z. For practical purposes, the access envelope AE may define regions through which the ablation device 10 may successfully deliver high frequency signals through the supply electrode 12 to provide therapeutic treatments. Each of the dimensions are generally described in reference to the longitudinal axis A_{L} in order to demonstrate the variations or divergence of portions of the ablation device 10 from the longitudinal axis A_{L}. The divergence from the longitudinal axis A_{L} may simulate an entry or access path and corresponding passage necessary to access a region of interest. Additionally, the comparison of the dimensions of the ablation device 10 to the longitudinal axis A_{L} may illustrate how proportions of various curved devices may not be implemented in similar applications due considerable variation from the linear path of the longitudinal axis A_{L}.

Each of the dimensions associated with the ablation device 10 may be proportioned based on the dimensional requirements for a surgical application. However, the relative proportions, particularly between the depth Z, the width W, and the limited divergence from the longitudinal axis A_{L}, are provided by the relationship among the features forming the acting end 54, including the supply electrode 12, the return electrode 14, the insulator 16, as well as the transition passage 44 in connection with the lumen 20. In some implementations, the depth Z of the ablation device 10 may be less than 50% larger than the width W. In some examples, the depth Z may be less than 40%, 30%, and even less than 20% larger than the width W as defined by the outer wall 18a of the shaft 18. As depicted in FIG. 7B and described in the exemplary embodiments, the depth Z is approximately 18% larger than the width W. These dimensions are particularly important because a lower ratio of the depth Z to the width W may provide for the ablation device 10 to access target regions within the anatomy of a patient through a smaller access envelope AE. As demonstrated most clearly in FIG. 7B, the access envelope AE is defined as an elongated rectangle with a base having the width W and the height associated with the depth Z. For ease of reference, the access envelope AE may fit through a linear longitudinal path (e.g., the longitudinal access A L) along a circular cross section to define a cylindrical volume forming the access envelope AE having a diameter of approximately 1.2 times the depth Z or 1.4 times the width W. Such a narrow access envelope AE presents difficulties in facilitating the lateral alignment of the face 24 of the supply electrode 12 to the lumen 20 without interfering with the flow path FP and causing conditions susceptible to clogging. However, the access envelope AE defined in FIGS. 7A and 7B is accessible by the ablation device 10 while maintaining the improved performance of the transition passage 44 to limit turbulent flow conditions and improve access within a joint space or cavity of a patient with the supply electrode 12 implemented at the distal extent 34 of the tapered end portion 32. Accordingly, the ablation device 10 may provide for improved access and operation while maintaining a utility for the application of ablation therapy perpendicular to a longitudinal axis A_{L} of a device having an elongated supply shaft 18.

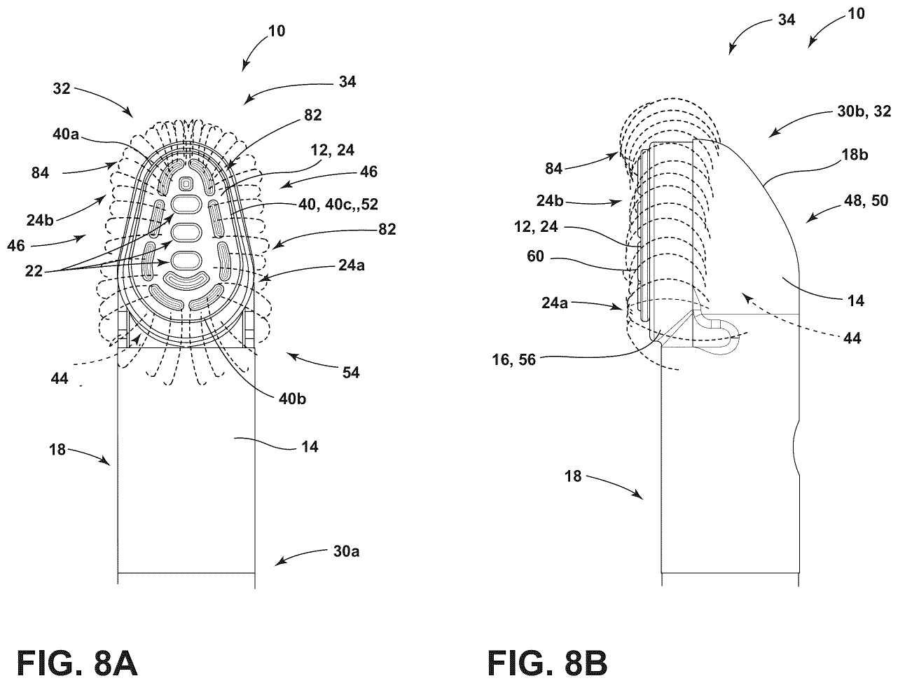

Referring now to FIGS. 8A and 8B, the spatial relationship among the active electrode 12, the insulator 16, and the return electrode 14 may provide for the generation of an ablation field 82 that extends over the electrode face 24 and about the perimeter edge 52. As described herein, the ablation field 82 may correspond to regions proximate to the acting end 54 over which the current density transmitted between the supply electrode 12 and the return electrode 14 is sufficient to effectuate ablation treatment. The transmission of the current and the resulting current density and distribution between the supply electrode 12 and the return electrode 14 may vary as a result of the power supplied from a controller or control console over a range of power settings (e.g., approximately 40 W+/−20% to 400 W+/−20% with a load of 220Ω). Accordingly, in response to an activation signal in the form of a high frequency alternating current (e.g., a radio frequency signal), the ablation field 82 may be induced between the supply electrode 12 and the return electrode 14 to provide an effective ablation treatment region over the surface of the electrode face 24. Additionally, the ablation field 82 and the effective treatment region may extend beyond the electrode face 24 about the perimeter edge 52, as shown in FIGS. 8A and 8B. As discussed further in the following examples, the region of the ablation field 82 extending beyond the perimeter edge 52 of the supply electrode 12 may be referred to as an edge ablation region 84.

In some implementations, the intensity of the ablation field 82 may be uniformly distributed about the perimeter edge 52 as a result of the geometry of the acting end 54, including the spacing between the supply electrode 12 and the return electrode 14. For example, as previously discussed, the consistent or approximately equidistant spacing along or parallel to the electrode face 24, denoted as S_{xy}, and the spacing perpendicular to the electrode face 24, denoted as S_{z}, may provide for distributed transmission of the current that drives or induces the ablation field 82 along the edge ablation region 84. As shown in FIGS. 8A and 8B, the effective range or reach of the ablation field 82 may extend about the perimeter 52 of the supply electrode 12 to the return electrode 14 along the first arc 40a and the opposing sides 46 of profile shape 40. In this configuration, the ablation device 10 may be implemented to apply an ablation treatment to manipulate tissue by maneuvering the opposing sides 46 of the acting end 54 proximate to a target or treatment region to ablate tissue along the edge ablation region 84.

In some examples, the edge ablation region 84 may be positioned proximate to tissue such that ablation therapy may be applied along the perimeter 52 of the electrode face 24 along the first arc 40a and the opposing or straight side portions 40c without effectuating ablation treatment to tissue over the electrode face 24. Accordingly, ablation energy may be selectively applied along the perimeter 52 by the edge ablation region 84. Additionally, ablation energy may be selectively applied over the electrode face 24. The determination of whether the ablation energy is delivered along the electrode face 24, the perimeter edge 52, or both regions may be controlled by maneuvering the acting end 54 to adjust the proximity of the opposing sides 46 or the electrode face 24 proximate to the target or treatment region. For example, if the opposing sides 46 are arranged closer to tissue than the electrode face 24, the ablation energy may act on the tissue along the edge ablation region 84. Alternatively, if the opposing sides 46 are arranged further from the tissue than the electrode face 24, the ablation energy may act on the tissue along the electrode face 24. If both the electrode face 24 and the opposing sides 46 are similarly spaced from the target tissue, the ablation treatment may be applied to the tissue along the electrode face 24 and the side portions 46.

The edge ablation region 84 may additionally provide for the distal electrode portion 24b of the acting end 54 to extend into narrow cavities (e.g., joint spaces) that may not accommodate the comparatively enlarged proportions of the proximal electrode portion 24a. That is, the gradual narrowing of the torpedo-like, tapered end portion 32 may provide for the distal electrode portion 24b and the edge ablation region 84 to access spaces and apply ablation treatments therein. Additionally, the uniform spacing between the supply electrode 12 and the return electrode 14 about the perimeter 52 may ensure the edge ablation region 84 is evenly distributed in intensity. In this configuration, the effect of the edge ablation region 84 about the perimeter 52 of the electrode face 24 may be applied with consistent results and spacing from the target tissues as when applying ablation treatment with the ablation field extending over the electrode face 24. Such consistency may provide for reliable and consistent treatment results that improve the user experience and patient outcomes.

Referring now to FIG. 9, an ablation system 90 comprising the ablation device 10 in an operating configuration is shown. As previously discussed, the ablation device 10 may be in communication with a controller 92 that may control the delivery of signals to the supply electrode 12 and/or control a fluid flow rate (e.g., an aspiration rate) through the lumen 20. In operation, the controller 92 may receive inputs via a user interface 88, which may be distributed among a control unit 94 as well as one or more external control devices 96. The external control devices 96 may correspond to one or more electronic or electromechanical buttons, triggers, or pedals incorporated on a handle portion 98 of the grip of the ablation device 10, one or more foot pedals 100, and additional peripherals and devices communicatively connected to the control unit 94. The user interface 88 may include one or more switches, buttons, dials, and/or displays, which may include soft-key or touchscreen devices incorporated in a display device 102 (e.g., liquid crystal display [LCD], light emitting diode [LED] display, cathode ray tube [CRT], etc.). In response to inputs received from the user interface 88, the controller 92 may activate or adjusts the settings of the control signals communicated to the ablation device 10. The control signals generated by control unit 94 may be controlled by a signal generator 104 configured to generate periodic or RF signals that induce a treatment field (e.g., an electric or RF field) in response to control instructions (e.g., timing signals, amplification, etc.) communicated from the controller 92. The control signals may be communicated from the signal generator 104 of the control unit 94 to the ablation device 10 via one or more conductive connectors 106.

The conductive connectors 106 may be connected to the active or supply electrode 12 to transmit the output control signal Tx and connected to the return electrode 14 to receive a return signal Rx. The return signal Rx may be monitored by the controller 92 to provide closed-loop feedback to adjust the control signal Tx. The control signal Tx from the signal generator 104 may correspond to an AC driving signal generated in response to time-modulated signals from a processor 110 of the controller 92. The AC driving signal may induce the treatment or ablation field in the form of RF energy. The modes of operation of the ablation device 10 may be controlled by adjusting the amplitude of the voltage and timing of the signal modulation that instructs the signal generator 104 to generate RF signals. Accordingly, by adjusting the voltage potential and the frequency or timing characteristics of the AC driving signal output from the signal generator 104, the controller 92 may control the operation of the ablation device 10 in response to inputs received via the user interface 88. In some embodiments, the controller 92 may be configured to activate one or more preset modes (e.g., ablation, coagulation) and the associated power levels or frequencies as presets in response to inputs received from the user interface 88.

The processor 110 of the controller 92 may be implemented as a microprocessor, microcontroller, application-specific integrated circuit (ASIC), or other circuitry configured to perform instructions, computations, and control various input/output signals to control the ablation system 90. The instructions and/or control routines 112 of the system 90 may be accessed by the processor 110 via a memory 114. The memory 114 may comprise random access memory (RAM), read only memory (ROM), flash memory, hard disk storage, solid state drive memory, etc. The controller 92 may incorporate additional communication circuits or input/output circuitry. In an exemplary embodiment, a communication interface 116 of the controller 92, may include digital-to-analog converters, analog-to-digital converters, digital inputs and outputs, as well as one or more peripheral communication interfaces or busses. The peripheral communication interfaces of the communication interface 116 may be implemented with by various communication protocols, such as serial communication (e.g., CAN bus, I2C, etc.), parallel communication, network communication (e.g., RS232, RS485, Ethernet), wireless network communication (Wi-Fi, 802.11, etc.). In some examples, the controller 92 may be in communication with one or more external devices 118 (e.g., control devices, peripherals, servers, etc.) via the communication interface 116. Accordingly, the control unit 94 may provide for communication with various devices to update, maintain, and control the operation of the ablation system 90.

Though not pictorially illustrated in the figures, a pump 120 or aspiration pump may be connected via one or more fluid conduits in connection with the lumen 20 to effectuate fluid transfer via a fluid transmission path FP comprising the aspiration aperture(s) or port(s) 22, the transition passage 44, and the lumen 20. The pump 120 may be controlled via the user interface 88 of the controller 92 to adjust a flow rate or intensity of the fluid transfer. The pump 120 may be implemented with a variety of pumping technologies (e.g., peristaltic, reciprocating, etc.) and may vary in fluid transfer capacity based on the application of the ablation device 10.

According to some aspects of the disclosure, an ablation apparatus comprises an elongated shaft comprising a lumen extending along a longitudinal axis from a proximal end portion to a distal end portion, a supply electrode forming an electrode face directed laterally from the longitudinal axis at the distal end portion and forming an electrode face comprising at least one aperture in connection with the lumen, a return electrode extending along the distal end portion of the elongated shaft; and an insulator interposed between the supply electrode and the return electrode, the insulator forming a transition passage of the lumen interconnecting the at least one aperture to the lumen along an arcuate swept path.

According to various aspects, the disclosure may implement one or more of the following features or configurations in various combinations:
the elongated shaft extends to a distal extent of the ablation apparatus and the return electrode extends over a portion of the distal extent;
the transition passage forms an inlet flow cross section that decreases along the transition passage to outlet cross section in connection with the lumen;
a cross section of the internal passage forming the transition passage is swept from the longitudinal axis along the lumen to the at least one aperture extending laterally from the longitudinal axis;
the electrode face of the supply electrode forms a distal electrode portion that tapers outward from the longitudinal axis of the apparatus to a proximal end portion;
the elongated shaft forms a tapered end portion opposing the electrode face;
the elongated shaft tapers gradually along the longitudinal axis to a distal extent of the ablation apparatus on a first side opposing the electrode face and as well as a second side and a third side extending along opposing sides of the electrode face;
the tapered end portion taper at along a slope that increases with increasing proximity to the distal extent;
the supply electrode forms a perimeter edge adjacent to the insulator and extends from a proximal electrode portion to a distal electrode portion;
the perimeter edge is evenly spaced from the return electrode along the distal end portion;
the supply electrode extends approximately equidistant from the return electrode along at least a distal 25% of an electrode length Le of the supply electrode;
the approximately equidistant spacing between the supply electrode and the return electrode includes an average spacing that is evenly spaced on average over the distal end portion including variations in the perimeter edge of the supply electrode as well as a return edge of the return electrode;
the electrode face forms an ovular shape comprising a proximal electrode portion and a distal electrode portion;
a major axis of the ovular shape extends parallel to the longitudinal axis; and
the proximal electrode portion forms a first arc comprising a first radius and the distal electrode portion forms a second arc comprising a second radius, the first radius is greater than the second radius.

According to another aspect, a method is disclosed for delivering an ablation treatment comprising supplying a control signal to supply electrode of an ablation device, conducting the control signal through the supply electrode to a return electrode across an insulating gap, the insulating gap is approximately constant over a distal end portion of the supply electrode, and communicating fluid through a lumen extending through an elongated shaft of the ablation device.

According to various aspects of the invention, the disclosure many implement one or more of the following features or configurations in various combinations:
the communicating fluid through a lumen of the ablation device comprises communicating the fluid through at least one aspiration port extending laterally from the lumen and through the supply electrode;
steering the fluid along an arcuate path from the at least one aspiration port to the lumen;
the communicating of the fluid further comprises communicating the fluid through a decreasing cross-sectional area along the arcuate path from the supply electrode to the lumen; and/or
passing an acting end of the ablation device through a rigid cylindrical access envelope having a diameter and a length, wherein the diameter is less than two times a width of the elongated shaft and the length is at least two times the width.

According to yet another aspect of the invention, an ablation apparatus comprises an elongated shaft comprising a lumen extending along a longitudinal axis from a proximal end portion to a distal end portion, a supply electrode forming an electrode face directed laterally from the longitudinal axis at the distal end portion, the electrode face comprising at least one aperture in connection with the lumen, wherein the electrode face forms an ovular shape having a first major axis that extends parallel to the longitudinal axis, a proximal electrode portion forming a first arc comprising a first radius, and a distal electrode portion forming a second arc comprising a second radius, wherein the first radius is greater than the second radius, a return electrode extending along the distal end portion of the elongated shaft, and an insulator interposed between the supply electrode and the return electrode, the insulator forming a transition passage of the lumen interconnecting the at least one aperture to the lumen.

According to various aspects, the disclosure may implement one or more of the following features or configuration in various combinations:
the distal end portion of the ablation apparatus forms a torpedo shape that tapers to a distal extent of the ablation apparatus along opposing edges of electrode face and along a rear surface opposite the electrode face;
the electrode face comprises a plurality of protrusions extending along the first arc and the second arc and about the at least one aperture;
the protrusions protrude from the electrode face along a radius to a protrusion distance;
the protrusions form elongated ridges that extend parallel to the ovular shape of the electrode face;
at least one aperture comprises a plurality of apertures evenly distributed over an internal cross section of the transition passage in fluid communication the lumen; and
the plurality of apertures form ovular apertures having a second major axis that extends perpendicular to the first major axis of the supply electrode.
the transition passage forms an ovular opening that terminates at the supply electrode, the ovular opening comprises a major axis extending along the longitudinal axis;
the plurality of apertures are formed through the supply electrode and spaced along the longitudinal axis; and
the plurality of apertures form ovular apertures having a major axis that extends perpendicular to the longitudinal axis along the electrode face.

According to various aspects, the disclosure may provide an access envelope, particularly for applications passing through cannula but also percutaneously:
the elongated shaft extends along the longitudinal axis and comprises a lateral wall forming the lumen;
the lateral wall extends outward over a lateral distance of W/2 to a width W of the lateral wall;
an acting end of the ablation apparatus comprises the active electrode, the insulator, and the return electrode, the extents of which define an access envelope or minimum access cross section of the ablation apparatus along the longitudinal axis;
the acting end extends along the longitudinal axis and diverges from the longitudinal axis by less than 25% of the width of the elongated shaft;
the access envelope is defined as a volumetric path formed by a cross-sectional area of the distal end portion of the ablation apparatus extending along the longitudinal axis;
the access envelope fits within a cylindrical boundary having a diameter of less than 2(W), less than 1.8(W), less than 1.6(W), or even less than 1.5(W) down to approximately 1.4(W);
the ablation apparatus comprises a depth (Z) extending from the electrode face to an opposing side portion formed by the elongated shaft, wherein, the cross-sectional area fits within a cylindrical boundary less than 1.5Z);
the depth (Z) is less than 1.75 times the width W of the lateral wall and may be less than 1.5 times the width W, less than 1.2 times the width of the lateral wall down to approximately 1.18 times the width of the later wall; and/or
the ablation apparatus fits within a volumetric access envelope extending along the longitudinal axis having a cross-sectional area of 1.5 times an outside shaft diameter of the elongated shaft over a length of at least three times the outside shaft diameter.

It will be understood that any described processes or steps within described processes may be combined with other disclosed processes or steps to form structures within the scope of the present device. The exemplary structures and processes disclosed herein are for illustrative purposes and are not to be construed as limiting.

It is also to be understood that variations and modifications can be made on the aforementioned structures and methods without departing from the concepts of the present device, and further it is to be understood that such concepts are intended to be covered by the following claims unless these claims by their language expressly state otherwise.

The above description is considered that of the illustrated embodiments only. Modifications of the device will occur to those skilled in the art and to those who make or use the device. Therefore, it is understood that the embodiments shown in the drawings and described above are merely for illustrative purposes and not intended to limit the scope of the device, which is defined by the following claims as interpreted according to the principles of patent law, including the Doctrine of Equivalents

1. An ablation apparatus comprising:
an elongated shaft comprising a lumen extending along a longitudinal axis from a proximal end portion to a distal end portion;
a supply electrode forming an electrode face directed laterally from the longitudinal axis at the distal end portion, the electrode face comprising at least one aperture in connection with the lumen;
a return electrode extending along the distal end portion of the elongated shaft; and
an insulator interposed between the supply electrode and the return electrode, the insulator forming a transition passage of the lumen interconnecting the at least one aperture to the lumen along an arcuate swept path.

2. The apparatus according to claim 1, wherein the elongated shaft extends to a distal extent of the ablation apparatus and the return electrode extends over a portion of the distal extent.

3. The apparatus according to claim 1, wherein the transition passage forms an inlet cross section that decreases along the transition passage to an outlet cross section in connection with the lumen.

4. The apparatus according to claim 1, wherein a cross section of the internal passage forming the transition passage is swept from the longitudinal axis along the lumen to the at least one aperture extending laterally from the longitudinal axis.

5. The apparatus according to claim 1, wherein the electrode face of the supply electrode forms a distal electrode portion that tapers outward from the longitudinal axis of the apparatus to a proximal end portion.

6. The apparatus according to claim 5, wherein the elongated shaft forms a tapered end portion opposing the electrode face.

7. The apparatus according to claim 6, wherein the elongated shaft tapers gradually along the longitudinal axis to a distal extent of the ablation apparatus on a first side opposing the electrode face as well as a second side and a third side extending along opposing sides of the electrode face.

8. The apparatus according to claim 7, wherein the tapered end portion tapers along a slope that increases with increasing proximity to the distal extent.

9. The apparatus according to claim 1, wherein the supply electrode forms a perimeter edge adjacent to the insulator and extends from a proximal electrode portion to a distal electrode portion, wherein the perimeter edge is evenly spaced from the return electrode along the distal end portion.

10. The apparatus according to claim 1, wherein the supply electrode extends approximately equidistant from the return electrode along at least a distal 25% of an electrode length Lₑ of the supply electrode.

11. The apparatus according to claim 10, wherein the approximately equidistant spacing between the supply electrode and the return electrode includes an average spacing that is evenly spaced on average over the distal end portion including variations in a perimeter edge of the supply electrode and a return edge of the return electrode.

12. The apparatus according to claim 1, wherein the electrode face forms an ovular shape comprising a proximal electrode portion and a distal electrode portion, and a major axis of the ovular shape extends parallel to the longitudinal axis.

13. The apparatus according to claim 12, wherein the proximal electrode portion forms a first arc comprising a first radius and the distal electrode portion forms a second arc comprising a second radius, wherein the first radius is greater than the second radius.

14. A method for delivering an ablation treatment comprising:
supplying a control signal to supply electrode of an ablation device;
conducting the control signal through the supply electrode to a return electrode across an insulating gap, wherein the insulating gap is approximately constant over a distal end portion of the supply electrode and conducting the control signal across an insulating gap generates an edge ablation region extending about the distal end portion of the ablation device between the supply electrode and the return electrode; and
communicating fluid through a lumen extending through an elongated shaft of the ablation device.

15. The method according to claim 14, wherein the communicating fluid through a lumen of the ablation device comprises:
communicating the fluid through at least one aspiration port extending laterally from the lumen and through the supply electrode

16. The method according to claim 15, wherein the communicating fluid through a lumen of the ablation device further comprises:
steering the fluid along an arcuate path from the at least one aspiration port to the lumen.

17. The method according to claim 16, wherein the communicating of the fluid further comprises communicating the fluid through a decreasing cross-sectional area along the arcuate path from the supply electrode to the lumen.

18. The method according to claim 14, further comprising:
passing an acting end of the ablation device through a rigid cylindrical access envelope having a diameter and a length, wherein the diameter is less than two times a width of the elongated shaft and the length is at least two times the width.

19. An ablation apparatus comprising:
an elongated shaft comprising a lumen extending along a longitudinal axis from a proximal end portion to a distal end portion;
a supply electrode forming an electrode face directed laterally from the longitudinal axis at the distal end portion and comprising at least one aperture in connection with the lumen, the electrode face having an ovular shape comprising:
a first major axis parallel to the longitudinal axis;
a proximal electrode portion forming a first arc comprising a first radius; and
a distal electrode portion forming a second arc comprising a second radius, wherein the first radius is greater than the second radius;
a return electrode extending along the distal end portion of the elongated shaft; and
an insulator interposed between the supply electrode and the return electrode, the insulator forming a transition passage of the lumen interconnecting the at least one aperture to the lumen.

20. The apparatus according to claim 19, wherein the distal end portion of the ablation apparatus forms a torpedo shape that tapers to a distal extent of the ablation apparatus along opposing edges of electrode face and along a rear surface opposite the electrode face.
